(19) United States
(12) Patent Application Publication
(10) Pub. No.: US 2009/0209897 A1
(43) Pub. Date: Aug. 20, 2009

(54) Photoactivated Antimicrobial Wound Dressing and Method Relating Thereto

(75) Inventors: Santosh Y. Limaye, El Cajon, CA (US); Shanthi Subramanian, Princeton, NJ (US); Barbara R. Evans, Oak Ridge, TN (US); Hugh M. O'Neill, Knoxville, TN (US)

Correspondence Address: JOY L BRYANT, P.C., P O BOX 620, LIGHTFOOT, VA, 23090-0620, US

(73) Assignees: LOTEC, INC. DBA VESTA SCIENCES, INC., Santee, CA (US); UT-BATTELLE, LLC, Oak Ridge, TN (US)

(21) Appl. No.: 12/034,629

(22) Filed: Feb. 20, 2008

(51) Int. Cl. A61F 13/00

(52) U.S. Cl. 602/48

(57) ABSTRACT

A photoactivated antimicrobial wound dressing comprising a photocatalytic membrane is provided. The photocatalytic membrane comprises a bacterial cellulose hydrogel membrane having photocatalytic particles are immobilized within the membrane and are activated when exposed to light, at which time they react with oxygen-based species forming reactive oxygen species. The reactive oxygen species further react with microbes to kill the microbes.

### STATEMENT REGARDING FEDERALLY SPONSORED RESEARCH OR DEVELOPMENT

This invention was made with government support under Contract No. 1 R43 AI 65008-01A1 awarded by National Institutes of Health, National Institute of Allergy and Infectious Diseases. The Government has certain rights in the invention.

### FIELD OF THE INVENTION

The present invention is related to antimicrobial wound dressings. In particular, it is related to a photo-activated antimicrobial wound dressing.

### BACKGROUND OF THE INVENTION

The global wound care market encompasses millions of people who are treated for skin ulcers and burns. Wound healing resulting from trauma or surgical incisions usually proceeds over a reasonable length of time in most individuals. In contrast, compromised wounds such as pressure sores and diabetic ulcers tend to be chronic wounds that fail to heal through normal processes. Though these types of wounds initially involve the superficial skin, they eventually lead to erosion of the deeper layers of skin and underlying tissue. Any break in the skin gives bodily access to foreign pathogens, providing a fertile breeding ground and a potential site for serious infection. Preventing infection is critical to the successful healing of any wound, since infection not only lengthens healing time and treatment costs, but also can cause serious, and sometimes life-threatening, complications. In addition, many chronic wound patients have low immunity, thus lowering their body's ability to respond to bacterial invasion. This may be due to systemic diseases, malnutrition, immuno-suppressant therapy or advanced age, which makes preventing infection paramount to improved wound healing. With an aging population, cost-effective treatments for chronic wounds are critical.

As the understanding of the wound healing processes advances, many products are being developed to improve the basic steps of wound dressing and infection prevention. Dressings that provide a moist environment for wound healing have been proven to aid in the treatment of chronic wounds by donating moisture to the wound as well as absorbing exudate from the wound. Hydrocolloids, hydrogels and alginates are examples of these moist dressings. Hydrocolloids and alginates are absorbent and are used to absorb wound exudate. However, hydrocolloids are typically not recommended when the wound is infected and can sometimes break down. Hydrogels are not very absorbent but are effective in maintaining moist environments in dry wounds and provide a soothing effect. (Debbie Sharman, “Moist Wound Healing: A review of evidence, application and outcome,” The Diabetic Foot, 2003, Vol. 6, No. 3.) Bacterial cellulose based dressings have been shown to have better fluid management properties compared to other moist dressings because they have both fluid donation and absorption capabilities.

The use of additives in wound dressings to impart antimicrobial activity has been explored in various products. Broad spectrum antimicrobial agents such as chlorhexidine gluconate, benzalkonium chloride, parabens, polyhexamethylene biguanide (PHMB) and silver have been incorporated in wound dressings extensively to address the issue of infection. Silver based antimicrobials are very effective and many products are on the market with different silver compounds and release kinetics. These dressings require the continuous release of silver ions to be effective. The effect of prolonged release of silver into the wounds is unknown. There is also a concern of bacterial resistance to silver and consequently cross-resistance to antibiotics with the rise in use of silver in wound care (S. L. Percival et al., “Bacterial Resistance to Silver in Wound Care,” Journal of Hospital Infection, 2005, pp. 1-7, Vol. 60 and Simon Silver, “Bacterial Silver Resistance: Molecular biology and uses and misuses of silver compounds, “FEMS Microbiology Reviews, 2003, pp. 341-353, Vol. 27). Antiseptic agents such as PHMB have also been incorporated into bacterial cellulose as well as other types of dressings. Dressings containing PHMB are an effective barrier against entry of bacteria into the wound and also prevent bacterial growth in the dressing itself (Alejandro Cazzaniga et al., “The Effect of an Antimicrobial Gauze Dressing Impregnated with 0.2 Percent Polyhexamethylene Biguanide as a Barrier to Prevent Pseudomonas aeruginosa Wound Invasion,” Wounds: A compendium of clinical research and practice, June 2002, pp. 169-176, Vol. 14, No. 5). All these methods involve release of a foreign compound into the body and can lead to skin irritation and sensitization at higher concentrations.

Cellulose is the main component of plant cell walls and occurs in pure form in the cotton ball. More recently, it has been found that another form of cellulose is produced by certain acetic acid bacteria (Acetobacter strains). Although chemically identical, the physical properties of this bacterial cellulose differ greatly from those of the cellulose produced by green plants. Plant cellulose has a fibrous structure, while bacterial cellulose (BC) resembles a gel, containing over one hundred times its weight in water in its natural hydrated state. The differences in the properties of these materials result from their nanoscale structural architecture. Cellulose that is synthesized by plants has a structure composed of parallel macrofibrils that have a diameter ranging from 30 to 360 nm. The macrofibrils are then assembled into fibers. In contrast, bacterial cellulose is secreted in the form of small macrofibrils 40 to 60 nm wide that form a net- or sponge-like porous matrix in a centimeter-thick layer called a pellicule on the surface of the culture medium. When dried, the pellicule forms a thin, paper-like flexible membrane.

The cellulose secreted by Acetobacter strains exhibits unique physical, chemical and mechanical properties including high crystallinity, a large capacity to hold water, large surface area, elasticity, mechanical strength when wet and biocompatibility. This material has been proven to be effective as a dressing in wound care because of its biocompatible nanostructure, the ability to retain water, which provides a moist environment for wound healing and easy release of the dressing from the affected site. Wound care products based on bacterial cellulose are marketed as Biofill™ and Xcell™ dressings which are commercially available from Biofill Productos Biotechnologicos, Curritiba, Parana, Brazil and Xylos Corporation.

U.S. Pat. Nos. 4,588,400; 4,655,758; and 4,788,146 to Ring et al. disclose the use of microbial derived cellulose in liquid loaded medical pads. However, these patents do not disclose combinations of antimicrobial agents and cellulose, nor do they disclose a photoactivated antimicrobial wound dressing. U.S. Pat. No. 4,912,049 to Luiz F. X. Farah discusses the use of bacterial cellulose as artifical skin but fails to disclose a photoactivated antimicrobial wound dressing.

U.S. Patent Publication 2004/0028722 to Serafica et al. discloses a microbial cellulose wound dressing for treating chronic wounds. In this publication, a wound dressing comprising a microbial derived cellulose with an antimicrobial additive is disclosed. The antimicrobial additive in this case is the chemical compound polyhexamethylene biguanide. (PHMB) which is not a photoactivated compound. U.S. Patent Publication 2004/0142019 also to Serafica et al. discusses a microbial-derived cellulose amorphous hydrogel wound dressing. This publication also describes the addition of active agents such as antibiotics, silver-based antimicrobials, copper-based antimicrobials, anti-virals and anti-fungals in the microbial cellulose dressings. However, neither of these publications teaches a photoactivated antimicrobial wound dressing based on bacterial cellulose.

Photocatalytic oxidation using metal oxide catalysts (especially TiO₂) has been extensively investigated for the oxidation of organics in water and air, recovery of metals from effluents, and as antibacterial agents. Photocatalytic disinfection using TiO₂ has been investigated for a number of organisms including bacteria, viruses and yeasts in aqueous environments. In photocatalysis, incident ultraviolet (UV) light creates electron-hole pairs in a semiconductor catalyst. The charge carriers can react with adsorbed electron donors or acceptors on the surface of the catalyst resulting in the formation of the following species: hydroxyl radical, hydrogen peroxide and superoxide, in aqueous environments. (Blake et al., “Application of the Photocatalytic Chemistry of Titanium Dioxide to Disinfection and the Killing of Cancer Cells,” Separation and Purification Methods, 1999, pp. 1-50, Vol. 28, No. 1.) This reaction is demonstrated by the following mechanism:

### Electron-Hole Pair Formation:

TiO₂+hv→TiO₂⁻+OH. (or TiO₂⁺) (Conduction band electron and valence band hole)

Upon production on the TiO₂ surface, both hydroxyl radicals and/or superoxide have to react with the outer surface of the organism. Hydrogen peroxide can also penetrate the cell wall and result in hydroxyl radicals by reacting with Fe²⁺ ions via the Fenton reaction:

Fe²⁺+H₂O₂→OH.+OH⁻¹+Fe³⁺

There have also been efforts to create titania coatings on cotton fabrics to create antibacterial finishes. Daoud and Xin (“Nucleation and Growth of Anatase Crystallites on Cotton Fabrics at Low Temperatures,” J. Am. Ceram. Soc., 2004, pp. 953-955, Vol. 87, No. 5.) describe a coated cotton fabric with TiO₂ nanocrystals using a sol-gel process. The coating process was carried out on dry cotton fabric and requires hydrothermal processing to convert the titania gel into crystals.

Zhang and Qi (“Synthesis of Mesoporous Titania Networks Consisting of Anatase Nanowires by Templating of Bacterial Cellulose Membranes,” Chem. Commun., 2005, pp. 2735-2737.) have used bacterial cellulose as a template to grow titania nanowires. In this case, composites are formed by depositing titania gels within the pores of bacterial cellulose. In order to form porous, crystalline titania nanowires which are photocatalytic, the process requires that the composites be calcined at 500° C. Zhang and Qi note in their publication that the bacterial cellulose template started pyrolyzing at approximately 280° C. and was completely pyrolyzed by 500° C. Therefore, they fail to teach the combination of a hydrogel bacterial cellulose membrane and photocatalytic particles, or more specifically, crystalline titania. Rather, it is disclosed that in making photocatalytic titania nanowires, the cellulose is removed due to pyrolysis.

Huang et al. (“Biomolecular Modification of Hierarchical Cellulose Fibers through Titania Nanocoating,” Angew. Chem. Int. Ed., 2006, pp. 1-5, Vol. 45.) have also coated cellulosic fibers with titania gel for biofunctionalization. To coat the nanofibers with titania gel layers, a piece of filter paper was placed in a suction filtering unit, washed with ethanol, and dried by aspirating the air. A solution of Ti(OnBu)₄ in toluene/ethanol was then passed through the filter paper in 2 min. Ethanol was immediately passed through the filter paper to remove the unreacted metal alkoxides, followed by water to promote hydrolysis and condensation. Finally, the filter paper was dried with a flow of air. By repeating these procedures, the surfaces of the individual cellulose nanofibers were coated with ultrathin titania gel. To verify the uniform coating, the filter paper was calcined and examined by electron microscopy.

An object of the present invention is to provide a wound dressing based on a photocatalytic membrane prepared from a bacterial cellulose hydrogel membrane which becomes antimicrobial when photoactivated. The photoactive properties of this material are derived from the incorporation of photocatalytic particles immobilized within the bacterial cellulose hydrogel membrane.

Another object of the present invention is to provide an antimicrobial wound dressing prepared from a photocatalytic membrane comprising a bacterial cellulose hydrogel membrane and photocatalytic particles which are immobilized in the membrane.

Another object of the present invention is to provide an antimicrobial wound dressing that, when exposed to light, reacts with oxygen-based species to form reactive oxygen species that react with microbes to kill the microbes.

Another object of the present invention is to provide a method of treating wounds using the photoactivated antimicrobial wound dressing.

### SUMMARY OF THE INVENTION

The present invention is for a photoactivated antimicrobial wound dressing comprising a photocatalytic membrane. The photocatalytic membrane comprises a bacterial cellulose hydrogel membrane having photocatalytic particles immobilized within the hydrogel membrane. The photocatalytic particles are activated when exposed to light, at which time electron-hole pairs are created in the particles, which react with oxygen-based species forming reactive oxygen species. The reactive oxygen species further react with microbes to kill the microbes.

A method for treating a wound using the photoactivated antimicrobial wound dressing of the present invention is also presented. In practicing the method, the photoactivated antimicrobial wound dressing is provided. The wound dressing is applied to a wound. The wound dressing is exposed to light wherein the photocatalytic particles react with oxygen-based species forming reactive oxygen species. The reactive oxygen species further react with microbes in the wound, killing the microbes.

Additional objects and advantages of the invention will be set forth in part in the description which follows, and in part, will be obvious from the description, or may be learned by practice of the invention. The objects and advantages of the invention will be obtained by means of instrumentalities in combinations particularly pointed out in the appended claims.

### BRIEF DESCRIPTION OF THE DRAWING

The accompanying drawing illustrates a complete embodiment of the invention according to the best modes so far devised for the practical application of the principals thereof, and in which:

FIG. 1 is a graph showing the phototoxicity of a membrane of the present invention on a normal fibroblast.

### DETAILED DESCRIPTION OF THE PREFERRED EMBODIMENTS

The present invention is directed towards a photoactivated antimicrobial wound dressing. For the purpose of this disclosure and the claims, photoactivated encompasses exposure to light of any wavelength. The microbes which are treated by the wound dressing of the present invention include but are not limited to: bacteria; bacterial spores; viruses; fungi; and yeasts. In general, the photoactivated antimicrobial wound dressing comprises a photocatalytic membrane. The photocatalytic membrane comprises a bacterial cellulose hydrogel membrane having photocatalytic particles immobilized therein.

There are several advantages to using a bacterial cellulose hydrogel membrane in the present invention. The presence of water in the bacterial cellulose membrane promotes the formation of hydroxyl radicals necessary for photocatalytic disinfection. In addition, the presence of water allows for the reactive oxygen species to diffuse through the membrane to the wound. The hydrogel is semi-transparent to UV light. As a result, photoactivation occurs throughout the membrane and is not restricted to the membrane surface.

The bacterial cellulose hydrogel membranes are prepared by cultivation of cellulose-producing bacteria. Any cellulose-producing bacteria known to one of ordinary skill in the art may be used. In particular, cellulose-producing bacteria of the species Gluconacetobacter hansenii or Gluconacetobacter xylinus (synonyms Acetobacter xylinus, Gluconoacetobacter xylinus, Gluconoacetobacter hansenii), or a combination consisting of the two species may be used. The bacteria are cultivated under static conditions in rich media formulations or with an improved synthetic medium. As one embodiment of the invention, the bacterial cellulose hydrogel membrane is chemically modified to enable the deposition of crystalline TiO₂, or other photocatalytic particles, maximizing the surface area available for photoactivation and increasing efficiency.

The photocatalytic membrane is prepared by incorporating photocatalytic particles with the bacterial cellulose hydrogel membrane. Any photocatalytic particles known to one of ordinary skill in the art may be used for the present invention. Examples of these particles include but are not limited to: TiO₂; ZnO; ZnS; α-Fe₂O₃; WO₃; SrTiO₃; K₄Nb₆O₁₇; and perovskite mixed oxides. The photocatalytic particles are typically nanometer-sized particles. Nanosized particles are expected to exhibit enhanced photocatalytic activity because of their increased surface area and unique photocatalytic properties. More specifically, the photocatalytic particles are crystalline titanium dioxide (TiO₂) nanoparticles having a particle size ranging from about 5 nm to about 100 nm. Most preferably, the crystalline TiO₂ nanoparticles are of the anatase form. The crystalline TiO₂ nanoparticles typically are loaded at a level ranging from about 0.5 mg/cm² to about 4 mg/cm² per unit area of the membrane, and most preferably are loaded at 3 mg/cm² per unit area of the membrane. The particles are loaded on either one or both sides of the membrane, preferably on both sides of the membrane.

Several methods are used to incorporate crystalline TiO₂ into the hydrogel membranes. Crystalline TiO₂ particles can be formed in situ in the membranes or preformed crystalline TiO₂ particles can be loaded into the membranes. The amount of crystalline TiO₂ can range from about 0.5 mg/cm² to about 4 mg/cm² per unit area of the membrane and most preferably is loaded at about 3 mg/cm² per unit area of the membrane. Crystalline TiO₂ particles can be loaded on one side or both sides of the membrane, but preferably on both sides. In this embodiment, preformed crystalline TiO₂ particles of precisely determined sizes are incorporated into the cellulose or other fabric matrix by vacuum filtration. Preformed crystalline TiO₂ particles of specific sizes are loaded into a hydrated cellulose membrane by placing the membrane on a filtration device and applying vacuum while a suspension of the required amount of crystalline TiO₂ particles is loaded on the top side of the cellulose. Once the infiltration is complete, the membrane is rehydrated, inverted and the appropriate amount of a crystalline TiO₂ particle suspension is filtered through the membrane from the second side.

In some instances, the hydroxyl groups in the cellulose hydrogel exhibit weak bonds to the photocatalytic particles, which help to immobilize the particles within the membrane. In one embodiment, pre-formed crystalline TiO₂ particles of precisely determined sizes are incorporated into the cellulose hydrogel membrane by diffusion.

In all instances, the photocatalytic particles are immobilized within, on, or near at least one surface of the bacterial cellulose hydrogel. This immobilization enables the photocatalytic particles to be easily removed from the wound because the particles remain within the membrane. If the photocatalytic particles were not bound within the wound dressing, then the particles would penetrate into the wound and likely interfere with the healing process. The immobilization is critical to allow for removal of the photocatalytic particles from the site of the wound.

The bacterial cellulose-crystalline TiO₂ membranes have a water content ranging from about 2% to about 98% by weight. The water content is adjusted by appropriately drying the membrane to the desired extent. Drying can be accomplished most preferably by using a vacuum assisted gel dryer. Other techniques such as air drying, mechanical pressing or centrifugal draining can also be used.

The membranes are packaged and stored in a variety of ways. In one embodiment, the membranes are steam sterilized after the desired water content is obtained. Other sterilization techniques commonly used for wound dressings such as gamma irradiation and electron beam sterilization are also used, once the dressing is packaged in a moisture impermeable package.

The antimicrobial wound dressing is activated when the photocatalytic membrane is exposed to light. By activated, it is meant that the photocatalytic particles undergo photocatalysis. In turn, following exposure to light, the photocatalyic particle can act as either an electron donor or acceptor for molecules in the surrounding media. Since there is oxygen present from water in the hydrogel, typically the photocatalytic particles react with the hydroxide ions to form a reactive oxygen species (or free radicals). This reactive oxygen species then reacts with microbes either destroying the cell wall or modifying the chemical nature of the microbe to kill the microbe.

The antimicrobial wound dressing is exposed to light in order to activate it. The light may be any light having a wavelength ranging from about 200 nm to about 800 nm. Preferably, the light is ultraviolet light having a wavelength of about 365 nm. Typically, the wound dressing is activated after it is applied to the wound. As an additional embodiment, once the dressing has been applied to the wound, a portion of the dressing may be covered by an overdressing in order to protect the photocatalytic membrane.

### EXAMPLES

### Example 1

### Growth of Bacterial Cellulose Membranes

The cellulose membranes are prepared by cultivation of cellulose-producing bacteria of the species Gluconacetobacter hansenii or Gluconacetobacter xylinus (synonyms Acetobacter xylinus, Gluconoacetobacter xylinus, Gluconoacetobacter hansenii), or a combination of the two species. The bacteria are cultivated under static conditions in rich media formulations or with an improved synthetic medium. For cellulose production, precultures of the bacteria are diluted ten-fold with fresh media. Culture is carried out at a temperature of 28-30° C. by adding an aliquot of activated seed broth to the culture medium (Iguchi2000). As observed in earlier studies, the cellulose pellicule begins to form at the surface of the media and its thickness increases with time. Culture dishes of 6 or 9 cm diameter are used for cellulose production. In the process of gel growth, the aerobic bacteria generate cellulose only in the vicinity of the surface, so that productivity depends primarily on the surface area, not on the volume of the vessel.

Once the growth is completed, the pellicule is harvested and undergoes a cleaning process. To remove the bacteria, the cellulose pellicules are first heated at 90° C. in distilled water, then treated with 1% sodium hydroxide for a few hours to a few days at 23° C. Several washes with 1% NaOH solution are required. The absorbance of the NaOH solution is monitored and the washes are continued until the absorbance at 280 nm is between 0.01 and 0.05. The hydroxide is neutralized by the addition of a slightly acidic solution (pH 4 to 5) of either acetic acid or sodium dihydrogen phosphate, followed by washing in deionized water to remove the sodium acetate or sodium phosphate. An alternate process for cleaning was to use several washes in deionized water until the pH is neutral. The processing here is necessary to convert the cellulose into a medical grade and non-pyrogenic wound dressing material and can take between 1 to 7 days depending on the thickness of the pellicule.

### Example 2

### Infiltration

Nanosized crystalline (anatase form) TiO₂ of two grades was obtained from Alfa Aesar. The first grade had a particle size of 32 nm with a surface area of 45 m²/g and the second grade had a particle size of 5 nm with a surface area of 200-220 m²/g. Since the photocatalytic activity increases with surface area, Grade 2 TiO₂ particles were expected to be more effective than Grade 1. A dispersion of crystalline TiO₂ particles with a concentration of 1 mg/ml was prepared by suspending 100 mg of crystalline TiO₂ in 100 ml dilute nitric acid solution (0.075M). The dispersion was sonicated in an ultrasonic bath for 10 minutes prior to incorporation to ensure that the crystalline TiO₂ particles were well dispersed and thus forming a TiO₂ suspension.

The cleaned bacterial cellulose (BC) membranes (9 cm in diameter) were placed in a vacuum filtration unit with a 90 mm diameter PES membrane. For a loading of ˜1.5 mg/cm² on one side, approximately 100 ml of the TiO₂ suspension was added to the unit immediately after sonication. A low vacuum was applied to the unit allowing the solution to permeate through the cellulose membrane. The membrane was removed after the permeation was complete and washed with deionized (DI) water and immersed in DI water at 85-90 C in a water bath. The TiO₂—BC membranes were autoclaved and stored in DI water.

For double sided loading, the membranes were immersed in DI water for periods ranging from about 1 hour to 24 hours, most preferably 24 hours, after one side to allow rehydration and swelling of the membrane. The membrane was then inverted and the procedure repeated with an additional 100 ml of the suspension to obtain loading of ˜3 mg/cm². Rehydration was found to help to increase the TiO₂ loading in the membrane.

The membranes were soaked in DI water for 24 hours, to ensure that they were pH neutral, and then were autoclaved in steam for sterilization and stored in sterile water.

### Example 3

### Photocatalytic Activity

The photocatalytic activity of the membranes of Example 2 was tested under UV illumination using the oxidation of a Procion Red Dye as a model reaction. The membranes were partially dried in a gel dryer to increase the cellulose content to 5-6% by weight. When the membranes were in contact with the dye and exposed to UV illumination with maximum emission at approximately 365 nm, photocatalytic oxidation was observed and resulted in reduced absorbance from the dye at a wavelength of 540 nm. A minimum intensity of 2000 μW/cm² as measured by a UVP J221 meter is needed to activate the membranes. The time of exposure ranged from 5 minutes to 120 minutes.

### Example 4

### Photocatalytic Activity of BC—TiO₂ composite membrane

The BC—TiO₂ composite membrane of Example 2 was placed in a 9 cm petri dish. 25 cc of a 2.5 wt % dye solution was added to the petri dish. For the one-sided membranes, the membrane was placed such that the TiO₂ side faced the UV lamp. A high intensity long wave UV Lamp, BP-100AP from UVP products with maximum emission at 365 nm was used for the dye oxidation studies. The incident UV intensity was measured by a UVP J221 UVA meter. The photocatalytic effect was determined by measuring the absorbance of the dye at 540 nm using a Gilford UV/VIS Spectrophotometer. The decrease in the absorbance was measured as function of TiO₂ loading, intensity, exposure time and type of TiO₂. (Grade 1 and Grade 2).

Table 1 summarizes the results from the photocatalytic dye oxidation studies on membranes impregnated with Grade 2 TiO₂ as function of exposure time and intensity.

TABLE 1
Photocatalytic dye oxidation studies on membranes impregnated
with Grade 2 TiO₂ as function of exposure time and intensity

| | | | | | Ratio |
|---|---|---|---|---|---|
| TiO₂ | | Expo- | | Absorb- | Absorbance |
| loading | UV | sure | Sample | ance | sample/ |
| (mg/cm²) | Intensity | Time | Absorbance | (untreated | untreated |
| One side | (μW/cm²) | (hours) | (at 540 nm) | dye) | dye |
| 0.75 | 12000 | 1 | 0.4077 | 0.61 | 0.66 |
| 0.75 | 12000 | 2 | 0.13695 | 0.57 | 0.24 |
| 0.75 | 7700 | 1 | 0.40705 | 0.62 | 0.66 |
| 0.75 | 7700 | 2 | 0.26035 | 0.62 | 0.42 |

The dye oxidation results indicate that the TiO₂ impregnated membranes show significant photocatalytic activity. The decrease in the absorbance increases with exposure time and intensity. A maximum reduction of 75% is observed for dye exposed to 12000 μW/cm² intensity for 2 h.

### Example 5

### Antibacterial Activity of Photoactivated TiO₂—BC Membranes

The antibacterial activity of the BC—TiO₂ membranes of Example 2 was tested under UV illumination using a modified strike through assay. Bacterial suspensions were placed on the membranes and illuminated with UV for predetermined times. The membranes were placed on agar plates and incubated to observe growth of surviving bacteria. The results were evaluated by comparison with appropriate controls to determine the % killing of bacteria in contact with the BC—TiO₂ membranes when exposed to UV activation.

The steps for the assay using E Coli as an example are as follows:

### Reagents:

Bacterial test strain
Dilution medium (M9 minimal medium or phosphate buffered saline)
Growth medium (LB or equivalent for E. coli)
Nutrient agar plates (LB agar or equivalent for E. coli)
Sterilized samples of cellulose and other test materials
1. Inoculate test strain for log phase cells. This can be done by mixing 5 ml growth medium with 0.2 ml of an overnight pre-culture, then incubating for 1-2 hours at 37° C.
Alternative 1: inoculate 5 ml nutrient medium with 0.2 ml of glycerol stock of bacterial strain and incubate 2 hours.
Alternative 2: inoculate 5 ml nutrient medium with inoculation loop (usually 10 microliters) and incubate for 4-6 hours at 37° C.
2. While bacteria are growing to log phase, samples are prepared of the material to be tested.
9-cm hydrated membranes are cut into 4 equal pieces.
Two identical quarter pieces, one for control and one for illumination, are needed for each condition.
3. Each quarter is weighed to obtain hydrated weight.
4. Water is removed from matched quarter pieces to obtain desired degree of hydration.
5. Each quarter is weighed to obtain dehydrated or dry weight.
6. The log phase culture is diluted with 1/100 with M9 medium (for example, 0.1 ml+10 ml M9).
The log phase cultures and all dilutions are mixed well immediately before pipetting to maintain the bacteria in a uniform suspension.
7. Each prepared quarter piece of cellulose is placed onto a nutrient agar plate.
8. 50 microliters of the 1/100 diluted log phase culture is pipetted on the middle of each piece.
9. Using the desired light source, each matched pair of cellulose pieces on nutrient agar plates is illuminated for the desired intensity and time. The controls are kept on the lab bench away from light.
10. From the remaining 1/100 dilution, 1/1000 dilution (100 microliters+1 ml M9) is prepared for determination of colony forming units (CFU). 50 and 100 microliters of the 1/1000 dilution are pipetted onto nutrient agar plates and spread with plastic or glass spreaders.
11. Once the illumination period is complete, all three sets of nutrient agar plates—control, illuminated, and CFU determination are put into the incubator at 37° C.
12. Bacterial growth is evaluated after 1 day and again after 2 days. Colonies on CFU plates are counted to determine number of CFU in assays.

The antibacterial activity was evaluated by the following a serial dilution method. The cellulose membranes were sterilized and the hydration state adjusted as described above for the strike through assay. The one-hundred fold dilution of the test microorganism was prepared and dilutions plated for CFU determination. Each sample piece of cellulose material was then placed in a 3.5 cm empty sterile culture dish and the 50 μl of microbial suspension were applied to each. The controls were kept covered while the experimental set was illuminated with the UV light for the desired time and intensity with the lids removed from the dishes. Then 2 ml of M9 CAA thia medium were added to each culture dish and they were incubated with gentle shaking at 23° C. for 1 h to detach the cells and allow partitioning between the cellulose surface and the liquid medium. Aliquots of the medium supernatant from each sample were used for serial dilutions with LB medium (Luria broth) with 0.1 ml of bacterial suspension mixed with 0.9 ml medium for each dilution. The serial dilution tubes were incubated for 1 day at the appropriate growth temperature for the test strain. Growth was first evaluated by visual examination of the turbidity of the serial dilution cultures, then confirmed by determination of the cell density as absorbance at 600 nm of the cultures in a Unicam UV-visible spectrophotometer against an LB medium blank. Aliquots of supernatant were also analyzed for viable cells by dilution and plating on nutrient agar for colony counts.

### Example 6

### Evaluation of Antibacterial Effect on Escherichia coli Using Serial Dilution

The effect of the amount of Grade 2 titania loaded on the membrane was evaluated by the serial dilution method (Table 2). Bacterial cellulose membranes loaded on both sides with approximately 1.5 mg/cm² and 3 mg/cm² Grade 2 TiO₂ were used for these tests. The illumination used was a XX15BLB lamp from UVP products with a shelf stand. The distance of the samples from the lamp was adjusted to vary the intensity. Samples were tested at 2400 μW/cm², and 5200 μW/cm² as measured by a UVP J 221 UVA meter. Increasing the loading of titania from 1.5 mg/cm² to 3 mg/cm² (double sided loading) on a 9-cm cellulose membrane was found to increase the photocatalytic killing of the bacteria by at least ten-fold. A 20 minute exposure at 5200 μW/cm² was sufficient to cause a 100 fold decrease in survival.

TABLE 2
Serial dilution assays were carried out as described above with 10⁴ CFU
E. coli K12 JM109 pACGFP applied to blank cellulose and titania-loaded
cellulose membranes.

| | % survival | | |
|---|---|---|---|
| Illumination | 2400 μW/cm² | 5200 μW/cm² | 5200 μW/cm² |
| conditions | 30 min | 20 min | 30 min |
| Blank | 100 | 100 | 100 |
| 1.5 mg/cm² titania | 100 | 100 | 10 |
| 3 mg/cm² titania | 10 | 1 | 1 |

### Example 7

### Effect on Micrococcus Luteus

Photocatalytic antimicrobial activity was also tested on a gram positive strain Micrococcus Luteus. Three samples: a control membrane with no TiO₂, a membrane with 1.5 mg/cm² TiO₂, and a membrane with 3 mg/cm TiO₂ (double sided loading) were used for these tests. The membranes were partially dried on a Savant Slab gel dryer to give 6 to 7 wt % cellulose content by weight before the experiments. The steps for the assay are as follows:

Micrococcus Luteus was inoculated and grown at 30° C. for 48 hours. The culture was diluted 50 fold with nutrient broth medium and grown at 30° C. for 6 hours. While bacteria were growing, the samples (hydrated membranes) were autoclaved for 15 min and cut into four equal pieces. Two pieces each of membranes with 0, 1.5 mg/cm² and 3 mg/cm² TiO₂ were placed on a nutrient agar plate. Four plates were prepared in total. 40 μl fresh culture was carefully pipetted on the middle of each membrane. Two sets of above nutrient agar plates were placed at room temperature away from illumination, and two other sets under a UV lamp. The plates were illuminated at an intensity of 6000 μW/cm² for 30 min.

Once the illumination period was complete, 1 set of nutrient agar plates, control and illuminated, were put into the incubator at 30° C. and incubated for 48 hours. The membranes from another set of plates were placed into 12 ml culture tubes with 2 ml nutrient broth medium and incubated at 37° C. in a incubator shaker. The bacterial growth was evaluated on the nutrient agar plates and the optical density (OD) at 600 nm was measured for bacterial growth in the nutrient broth medium after 48 hours.

The results from three experiments on growth inhibition in nutrient broth medium are tabulated below in Table 3. It is clear that there is significant inhibition of growth for the TiO₂ infused membranes when exposed to UV than without. The sample with approximately 3 mg/cm² TiO₂ loading shows higher inhibition compared to the 1.5 mg/cm² TiO₂ sample.

TABLE 3
Inhibition in nutrient broth medium for Micrococcus luteus for samples
with and without TiO₂ exposed to UV intensity of 6000 μW/cm²
for 30 minutes.

| TiO₂ | −UV/OD | +UV/OD | Growth Inhibition |
|---|---|---|---|
| 0 | 2.513 | 2.263 | 9.95% |
| 1.5 mg/cm² | 2.31 | 1.475 | 36.15% |
| 3 mg/cm² | 2.304 | 0.933 | 59.51% |

### Example 8

### Antifungal Activity

Testing was performed on Saccharomyces cerevisae ATCC 2601 yeast with UV illumination: light intensity 2147 μW/cm² at a wavelength of 365 nm in a ChromoVue cabinet.

Yeast was spotted onto the cellulose pieces then illuminated. The samples were shaken with liquid medium, then placed on agar plates. Plating aliquots of the liquid medium found that the yeast preferentially stayed stuck on the cellulose (few colonies grew up from plated liquid medium), regardless of whether it had titania in it or not (they even stay on after being killed by autoclaving). However, discrete colonies could be counted on the surface of the cellulose samples following placing on the agar and growth for 2 days.

TABLE 4
Colony counts on Top of Sample after 2 days incubation at 25° C.

| | | Grade 2 |
|---|---|---|
| Illumination time | | 1.5 mg/cm² loading |
| (min) | Blank | (both sides) |
| 0 | 39 | 48 |
| 15 | 42 | 42 |
| 30 | 44 | 19 |

The colony size was also smaller on 15 and 30 min exposed Grade 2 samples as compared to blanks (0.5 vs. 1 mm). These results show that at this intensity, Grade 2 TiO₂ loaded samples are effective in killing 60% of the yeast on exposure to UV illumination at 365 nm for 30 minutes. The killing rate can be improved by increasing the intensity and the TiO₂ loading.

### Example 9

### Cytotoxic Testing

The cytotoxicity of was evaluated by Fibroblast-Neutral Red Assay. A normal fibroblast cell line (CRL-1634) was purchased from ATCC and the cells were cultured in DMEM and expanded. The following procedure was used to determine phototoxicity as described by Lasarow et al., “Quantitative In Vitro Assessment of Phototoxicity by a Fibroblast Neutral-Red Assay,” Journal of Investigative Dermatology, 1992, pp. 725-729, Vol. 98.

Procedure for Assay:
1. Cells were seeded at a density of 1 million cells/ml in 0.5 ml/well of 24 well plate
2. Cells were incubated for 24 hrs
3. Neutral Red (NR) solution was prepared by diluting 0.4% aqueous neutral red stock solution 1:80 in DMEM, allowing insolubles to precipitate for 24 hr at RT (shielded from light). Centrifuging for 10 min at 1500 g gave the clear red supernatant for the assay
4. The next day, the positive control tetracycline was diluted in PBS (Ca2+ and Mg2− free) at concentrations of 1, 20, and 100 μg/ml
5. The culture medium was aspirated (12 wells, half plate) from the cells and replaced with 0.5 ml of PBS containing the diluted tetracycline. For each dose, 3 wells of cells were treated (triplicates).
6. The cells were incubated with the tetracycline for 30 min at 37° C. with 5% CO₂
7. Tetracycline containing PBS and the medium were aspirated from the other 12 wells and replaced with 0.5 ml of fresh PBS
8. BC—TiO₂ and blank membranes were placed into the cells.
9. The cells were exposed to UV light with maximum emission at 365 nm for a predetermined amount of time.
10. PBS was aspirated and the membrane were removed from the wells and replaced with 0.5 ml of NR-DMEM.
11. Cells were incubated for 3 hrs at 37° C. with 5% CO₂.
12. PBS was aspirated from the wells and cells were washed with 0.5 ml of an aqueous formal-Ca²⁺ solution to fix the cells for 1 min
13. The fixation solution was aspirated and replaced with an extraction solution containing 19% glacial acetic acid and 50% ethanol for 10 min
14. 0.15 ml of the extracted aliquot was transferred to wells of a 96 well plate
15. Absorption was measured at 540 nm with a plate reader
16. The phototoxic potential of a compound is expressed as the percentage decrease of NR uptake by cells in wells exposed to UVA with test compound, compared to that in wells exposed to UVA without the test compound

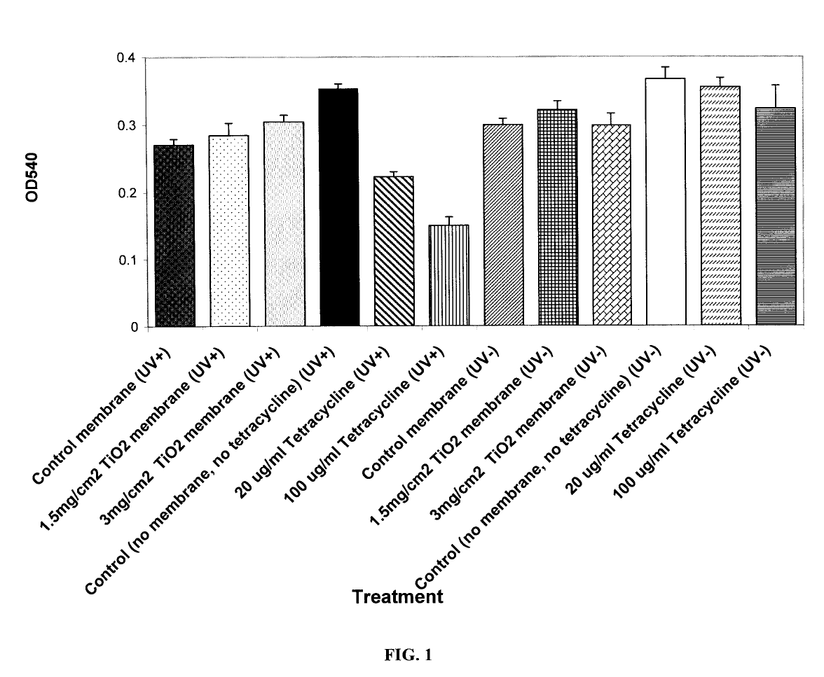

Results from this study are shown in FIG. 1. UV illumination of 6000 μwatts/cm² (as measured by UVP J221 meter) for 15 minutes from a XX15BLB fluorescent UVA lamp with maximum emission at 365 nm was used for the cytotoxicity study. The results from the study indicate that there is no difference between the UV treated membranes with and without TiO₂ (+) and the membranes not exposed to UV(−), suggesting that there is no toxicity due to the photocatalytic effect. All experiments with membranes show lower OD540 numbers with respect to the ones without membranes (labeled no treatment), because some cell adhesion to the membranes on removal from the wells is observed. There is no statistical difference between UV treatment and no UV treatment of the cells without the membrane. This indicates there is no cytotoxicity due to UV illumination at this intensity and exposure time. Tetracycline, which is used as a positive control, shows phototoxicity at the 100 μg/ml level.

The above description and drawings are only illustrative of preferred embodiments which achieve the objects, features and advantages of the present invention, and it is not intended that the present invention be limited thereto. Any modification of the present invention which comes within the spirit and scope of the following claims is considered part of the present invention.

What is claimed is:

1. A photoactivated antimicrobial wound dressing comprising:
a photocatalytic membrane comprising a bacterial cellulose hydrogel membrane having photocatalytic particles immobilized within the bacterial cellulose hydrogel membrane and wherein the photocatalytic particles when exposed to light react with oxygen-based species forming reactive oxygen species and wherein the reactive oxygen species react with microbes whereby the microbes are killed.

2. A photoactivated antimicrobial wound dressing according to claim 1, wherein the bacterial cellulose hydrogel membrane is prepared from at least one cellulose-producing bacteria.

3. A photoactivated antimicrobial wound dressing according to claim 2, wherein the cellulose-producing bacteria is selected from the group consisting of: Gluconacetobacter hansenii and Gluconacetobacter xylinus.

4. A photoactivated antimicrobial wound dressing according to claim 1, wherein the photocatalytic particles are crystalline titanium dioxide nanoparticles having a particle size ranging from about 5 nm to about 100 nm.

5. A photoactivated antimicrobial wound dressing according to claim 4, wherein the titanium dioxide nanoparticles comprise from about 0.5 mg/cm² to about 4 mg/cm² per unit area of a membrane.

6. A photoactivated antimicrobial wound dressing according to claim 5, wherein the crystalline titanium dioxide nanoparticles comprise about 3 mg/cm² per unit area of a membrane.

7. A photoactivated antimicrobial wound dressing according to claim 1, wherein the photocatalytic particles are incorporated into the bacterial cellulose hydrogel membrane by chemically modifying the bacterial cellulose to receive the photocatalytic particles.

8. A photoactivated antimicrobial wound dressing according to claim 1, wherein the photocatalytic membrane is activated by ultraviolet light having a wavelength of about 365 nm.

9. A photoactivated antimicrobial wound dressing according to claim 1, further comprising an overdressing wherein the overdressing covers at least a portion of the photocatalytic membrane.

10. A method for treating a wound, the method comprising the steps of:
a) providing a photoactivated antimicrobial wound dressing comprising a photocatalytic membrane comprising a bacterial cellulose hydrogel membrane having photocatalytic particles immobilized within the bacterial cellulose hydrogel membrane;
b) applying the photoactivated antimicrobial wound dressing to a wound; and
c) exposing the photoactivated antimicrobial wound dressing to light wherein the photocatalytic particles when exposed to light react with oxygen-based species forming reactive oxygen species and wherein the reactive oxygen species react with microbes whereby the microbes are killed.

11. A method according to claim 10, wherein the photocatalytic particles are crystalline titanium dioxide nanoparticles.

12. A method according to claim 11, wherein the photoactivated antimicrobial wound dressing is exposed to ultraviolet light having a wavelength ranging from about 250 nm to about 400 nm.

13. A method according to claim 10, further comprising the step of applying an overdressing over at least a portion of the photocatalytic membrane after the photocatalytic membrane has been exposed to light.
